(19) United States
(12) Patent Application Publication
(10) Pub. No.: US 2006/0292082 A1
(43) Pub. Date: Dec. 28, 2006

(54) Dry powder inhaler

(76) Inventors: Matthew Neil Sarkar, Cambridge, Cambridgeshire (GB); Carl Henry Pullen, Cottenham, Cambridgeshire (GB); Michael Edgar Newton, Diss, Norfolk (GB); Stephen William Eason, Diss (GB)

Correspondence Address: DAVIDSON, DAVIDSON & KAPPEL, LLC, 485 SEVENTH AVENUE, 14TH FLOOR, NEW YORK, NY, 10018, US

(21) Appl. No.: 10/571,512

(22) PCT Filed: Sep. 15, 2004
(86) PCT No.: PCT/GB04/03928

§ 371 Date: Jun. 5, 2006

(30) Foreign Application Priority Data
Sep. 15, 2003 (GB) 0321610.8

(51) Int. Cl. A61K 9/14; A61L 9/04; A61M 11/00

(52) U.S. Cl. 424/46; 128/200.23

(57) ABSTRACT

A dry powder inhaler for delivering a dose of medicament for inhalation by a user, including a drug entrainment device and a valve actuable by a user to cause pressurised gas to flow through a dose of medicament disposed in the drug entrainment device to entrain said dose in the gas, the valve comprising a valve member (28) configured such that, in a first mode, pressurised gas biases the valve member (28) into an open state to allow the flow of gas through the valve and, in a second mode, pressurised gas biases the valve member (28) into a closed state to prevent the flow of gas through the valve.

The present invention relates to an inhaler for the delivery of medicament in powdered form to the lung and more specifically to an active dry powder inhaler in which the medicament is entrained in a charge of pressurised gas or air to transport it from the medicament pack in which the dose is stored through the device and into the user's airway and down into the lungs.

Traditionally, inhalers have been used to deliver medicament to the lung to treat local diseases of the lung such as asthma. However, when the inhaled particles are in the range 1 to 3 microns they can reach the deep lung area (alveoli) and cross into the bloodstream. This systemic delivery of pharmaceutically active agents to the bloodstream via the lungs using an inhalation device has become a particularly attractive form of administering drugs to a patient many of whom are reluctant to receive drugs by injection using a needle. Furthermore, the administration of a drug using an inhaler may be carried out by a patient discreetly and in public without any of the known difficulties associated with injections involving a needle.

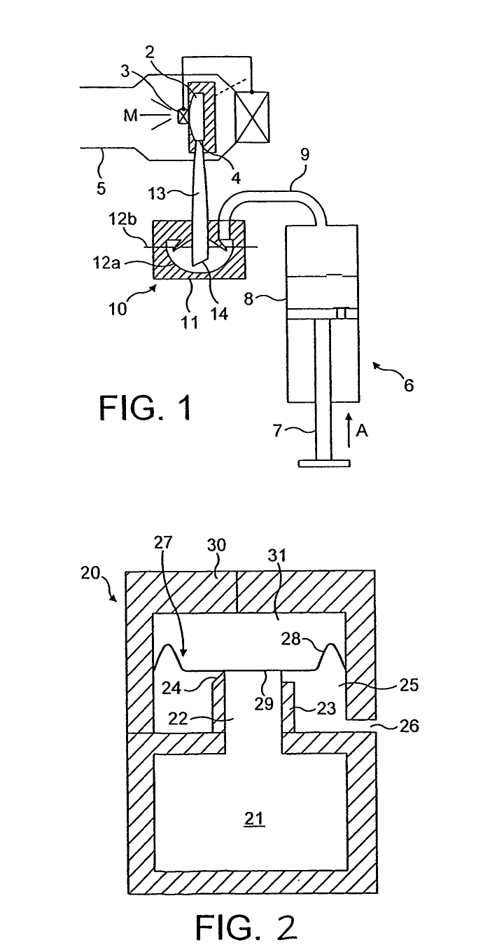

A schematic drawing of a conventional gas powered dry powder inhaler for aerosolising a powdered medicament for inhalation by a user is illustrated in FIG. 1. The inhaler 1 comprises a vortex chamber or nozzle 2 having an exit port 3 and an inlet port 4 for generating an aerosol of medicament M. The nozzle 2 is located within a mouthpiece 5 through which a user inhales the aerosolised medicament M. The dose is supplied to the nozzle 2 in an airflow generated by a pump represented in FIG. 1 as a piston pump 6 containing a plunger 7 received in a pump cylinder 8. An airflow path 9 extends from the pump cylinder 8 to a drug entrainment device 10 comprising a housing 11 to support a foil blister 12 containing a single dose of medicament (typically between 0.5 and 5 mg). The blister 12 has a cold-formed foil blister base 12a sealed with a hard rolled foil laminate lid 12b chosen to facilitate piercing. A drug feed tube 13 extends from the inlet port 4 of the nozzle 2 and into the housing 11 where it terminates in a piercing element 14. When the inhaler 1 is to be used, the pump 6 is primed with a charge of compressed air by sliding the plunger 7 into the pump cylinder 8 (in the direction of arrow “A” in FIG. 1) to compress the air contained therein. Thereafter, the housing 11 and the drug feed tube 13 and moved relative to each other to cause the piercing element 14 to break the foil laminate layer 12a and penetrate into the blister 12 so that when the user inhales through the mouthpiece 5 a valve 15, which may be breath actuated, releases the charge of compressed gas from the cylinder 8 so that it flows down the airflow path 9 into the blister 12 and up through the drug feed tube 13. As the air passes through the blister, the dose contained therein is entrained and is carried by the airflow up the drug feed tube 13 and through the inlet port 4 into the nozzle 2.

A rotating vortex of medicament and air is created in the nozzle 2 between the inlet and outlet ports 4,3. As the medicament passes through the nozzle 2, it is aerosolised by the high turbulent shear forces present in the boundary layer adjacent thereto as well as by the high level of turbulence in the vortex chamber and through collisions between agglomerates and other agglomerates and between agglomerates and the walls of the nozzle 2. The aerosolised dose of medicament and air exit the nozzle 2 via the exit port 3 and is inhaled by the user through the mouthpiece 5.

It will be appreciated that in an active inhaler of the aforementioned type, the same charge of gas provides the energy needed for both entraining the drug to evacuate the packaging and for aerosolising the drug once it has reached the nozzle. It is therefore important that as much as possible of the energy stored in the charge of gas is utilised to ensure efficient entrainment and aerosolisation of the dose and that restrictions to the flow of gas through the device are minimised. Bearing in mind that the amount of gas available for each dose is limited by what can be stored in a pressurised canister or generated in the device by a user by, for example, using a manually operated pump, the efficiency by which the drug is entrained in the airflow and so evacuated from its pack must be as high as possible.

To increase the efficiency of entrainment of the dose, it is important that the valve which releases the charge of compressed gas is opened quickly so that the charge enters the blister over a very short period of time and the dose receives sufficient fluid energy from the gas so that all or substantially all of the dose is entrained in the airflow. If the valve opens slowly, the dose will receive the charge of gas over a longer period with less energy and so some of the dose may not be entrained in the airflow resulting in a reduction in efficiency of the device.

It will be appreciated from the foregoing, that a valve is required that both opens rapidly and, presents a minimum resistance to flow once open. The speed by which a valve opens may be defined by the shortest time between the valve being fully closed and the valve being fully open. Additionally, it is also desirable that the forces required to operate the valve are as low as possible to reduce strain on components and facilitate ease of operation.

The effort required to keep a valve closed against a pressure is called the sealing force. The sealing force comprises two components: the pressure force Fₚ and the seat force Fₛ. The pressure force is the force generated by the pressure within a chamber and is given by the equation Fₚ=PA, where P is the pressure acting on the valve and A the area over which the pressure acts. Depending on the configuration of the valve, the pressure force may act to bias the valve towards the open or the closed position. The seat force, F_{s,} is the force required to create a continuous loop of intimate contact between the compliant part of the valve (the seal) and the valve seat.

An inhaler having a valve which is sealed by an immobilising mechanism and arranged so that the pressure acting on the valve acts to bias it towards an open position is known from U.S. Pat. No. 6,029,662. Although the valve opens rapidly because the compressed gas biases the valve to the open position and so assists opening, it is possible for the valve to leak because the closing mechanism has to oppose the pressure force generated in the chamber rather than use this pressure force to assist sealing. Therefore, in practice a high closing force to ensure sealing is required. A further disadvantage with this type of valve is that it must be re-set prior to re-pressurisation of the chamber.

To reduce the pressure force that must be overcome to seal the valve, the area of the valve exit orifice is minimised. However, this introduces the additional drawback that the speed of flow through the valve is considerably reduced so that although the valve opens rapidly, the speed at which the chamber empties is limited by the small size of the valve exit orifice.

In an alternative valve configuration, the pressure in the chamber biases the valve into a closed position to reduce the risk of leakage. The advantage of this approach is that the only force required to keep the valve closed is the seat force and this force may be provided by the pressure force. However, to open the valve, the pressure force acting on it must be overcome and this requires an actuation force much greater than the pressure force, especially if the valve is to be opened rapidly.

It will be appreciated from the foregoing that each of the above described types of valve embody an undesirable compromise. With a valve configuration of the first type, the valve opens rapidly but requires high forces to hold the valve closed and needs to be reset, for example by manually resetting. In the second case, the valve has a low closing force and can potentially be self-resetting, but a high opening force is needed for rapid opening.

The present invention seeks to provide a dry powder inhaler having a valve that overcomes or substantially alleviates the disadvantages associated with an inhaler having either of the types of valve described above.

According to the present invention, there is provided a dry powder inhaler for delivering a dose of medicament for inhalation by a user, including a drug entrainment device and a valve actuable by a user to cause pressurised gas to flow through a dose of medicament disposed in the drug entrainment device to entrain said dose in the gas, the valve comprising a valve member configured such that, in a first mode, pressurised gas biases the valve member into an open state to allow the flow of gas through the valve and, in a second mode, pressurised gas biases the valve member into a closed state to prevent the flow of gas through the valve. Although reference is made to pressurised gas, it should be understood that this includes compressed air in addition to gases.

Preferably, the valve is configured such that pressurised gas acts over both sides of the valve member when it is in the closed state. Although the pressure of the gas acting over each side of the valve member may be the same, it may act over a larger cross-sectional area of one side of the valve member than the pressurised gas acting over the other side of the valve member. This means that for the same given pressure, the force acting over a greater cross sectional area of the valve will be larger. As the force generated over one side of the valve member is larger, the valve member is maintained in a closed state.

In a preferred embodiment, the valve is configured such that the valve member moves from the closed state to the open state in response to a change in pressure of the gas acting on one side of the valve member relative to the pressure acting on the other side of the valve member.

The inhaler preferably comprises a reservoir for pressurised gas and a valve orifice for the passage of pressurised gas from the reservoir through the drug entrainment device. A first side of the valve member forms a seal with the valve orifice when in the closed state such that pressurised gas in said reservoir acts over only a portion of said first side of the valve member defined by the cross-sectional area of the valve orifice.

Conveniently, the valve orifice is located at the mouth of a tube in communication with the reservoir, the tube including a valve seat at the end thereof for cooperation with said first side of the valve member to form a seal therewith when the valve member is in the closed state.

The valve is preferably configured such that when the seal between the first side of the valve member and the valve seat is broken, the pressure of the gas in the reservoir acts over substantially the entire surface of the first side of the valve member to bias the valve member into the open state. As the pressure acting over one side of the valve is discharged, a threshold is reached at which the pressure of the gas in the reservoir acting over the other side of the valve is sufficient to cause the valve member to lift from the valve seat. When this occurs, the whole of the underside of the valve member is exposed to the pressure of the gas in the reservoir causing it to open rapidly.

In one embodiment the inhaler includes biasing means to bias the valve member into a closed state when the pressure of the gas in the reservoir has been discharged through the valve. This re-sets the valve member automatically into the closed state and removes any need to pressurise the other side of the valve member in advance of pressurisation of the reservoir.

The biasing means may conveniently comprise a spring.

In a preferred embodiment, means are provided to discharge the pressure that biases the valve member into the closed state to cause the valve member to move from the closed to the open state.

The valve preferably includes a primary chamber in which pressure to bias the valve member into the closed state is generated and said means for discharging the pressure that biases the valve member into the closed state comprises a discharge port in the primary chamber.

The valve advantageously includes means for opening the discharge port to atmosphere. Most advantageously, the means for opening the discharge port is breath actuated.

When the valve is breath actuated, it preferably includes a secondary valve member which is movable, in response to inhalation by a user, from a first closed position in which the discharge port is not in communication with the primary chamber to prevent discharge of the primary chamber to the atmosphere, into a second open position in which the discharge port is in communication with the primary chamber to discharge the primary chamber to the atmosphere.

The secondary valve member is preferably configured such that the pressure in the primary chamber acts over a smaller cross-sectional area of a first side of the secondary valve member than the cross-sectional area of the other side of the valve member over which atmospheric pressure acts, when the secondary valve member is in the closed position.

Conveniently, the valve member and secondary valve member may be flexible diaphragms.

The inhaler also preferably includes means for charging the reservoir with pressurised gas or air. Most preferably said means is also operable to charge the primary chamber.

A conduit may communicate the reservoir with the primary chamber to facilitate the charging of the primary chamber during charging of the reservoir with pressurised gas.

Embodiments of the invention will now be described, by way of example only, and with reference to FIGS. 2 to 9 of the accompanying drawings, in which:—

FIG. 1 is a schematic drawing of a conventional pressurised gas powered active dry powder inhaler;

FIG. 2 is a simplified cross-sectional side elevation of a valve assembly according to the invention;

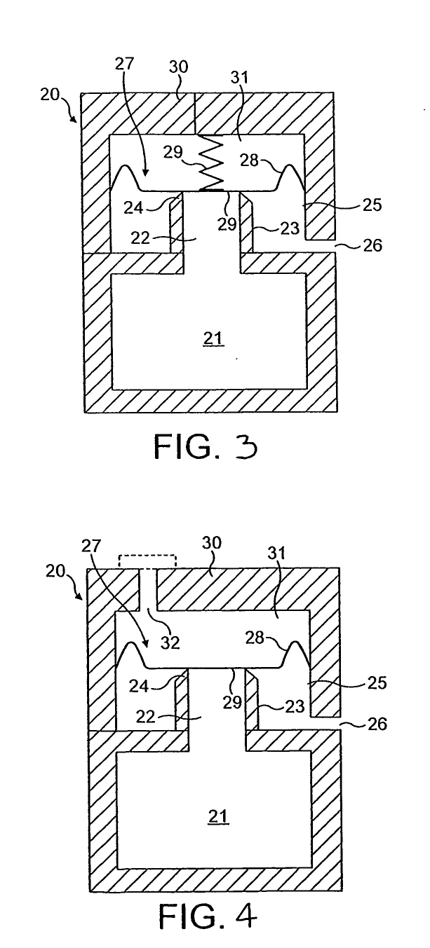

FIG. 3 is a first modified version of the valve assembly illustrated in FIG. 2;

FIG. 4 is second modified version of the valve assembly illustrated in FIG. 2;

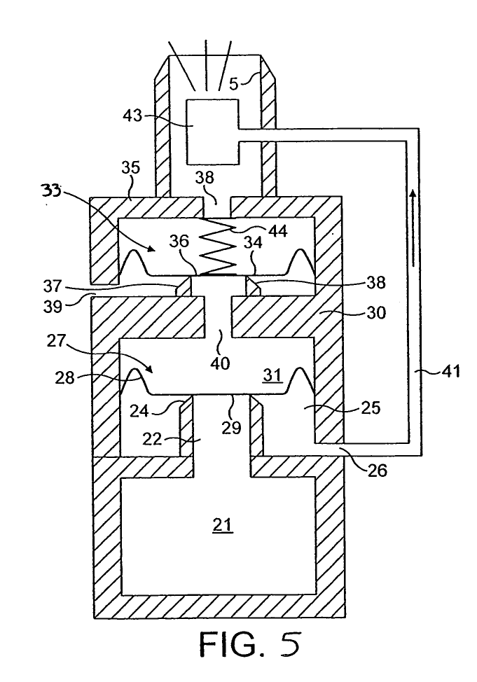

FIG. 5 is a third modified version of the valve assembly illustrated in FIG. 2;

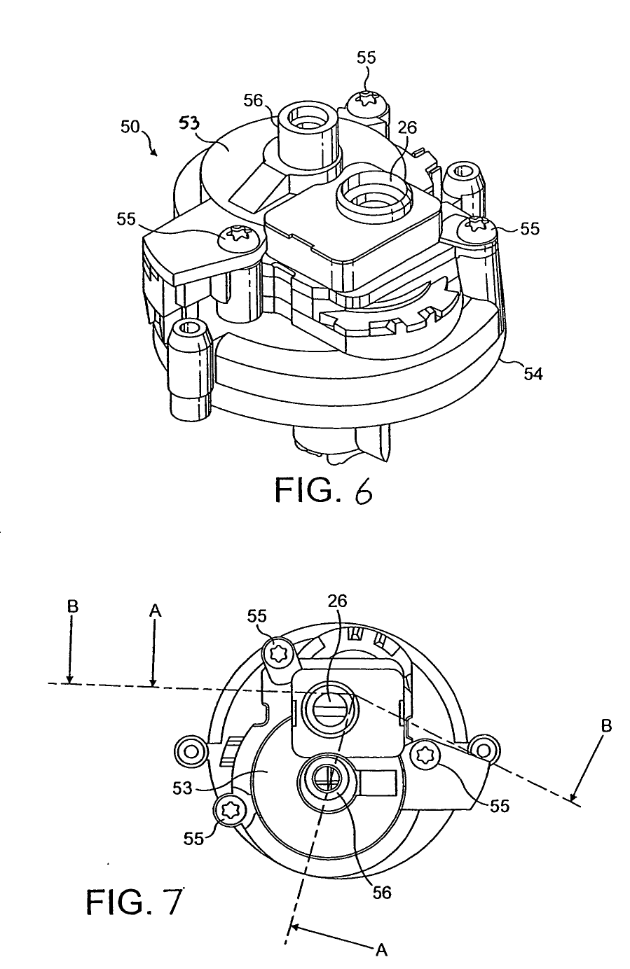

FIG. 6 is a perspective view of an actual breath actuated valve module forming part of an inhaler according to the invention;

FIG. 7 is top plan view of the breath actuated valve module shown in FIG. 6;

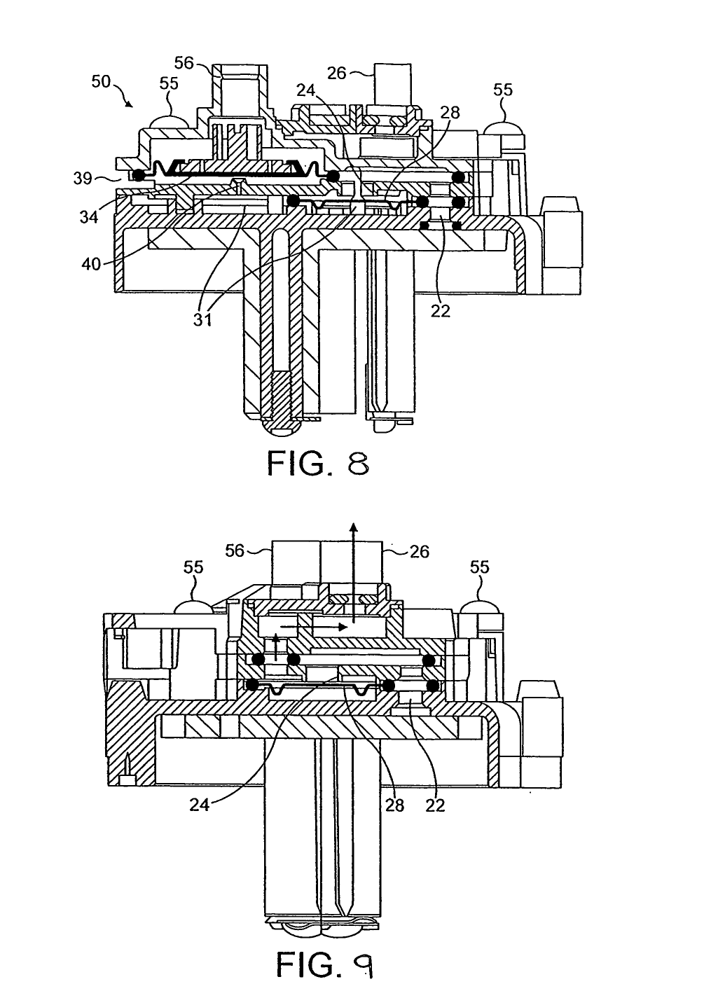

FIG. 8 is a cross-sectional side elevation of the breath actuated valve module taken along the section A-A in FIG. 7;

FIG. 9 is a cross-sectional side elevation of the breath actuated valve module taken along the section B-B in FIG. 7.

The conventional pressurised gas powered inhaler 1 of FIG. 1 has already been described in detail and so no further description of it will be made here. FIGS. 2 to 5 represent three highly simplified representations of valves that operate according to the principle of the invention and reference is first made to them for the purpose of explanation and to facilitate understanding of the invention.

Referring now to FIG. 2, there is shown an assembly 20 comprising a reservoir 21 containing a source of compressed gas or air. The reservoir 20 may be charged using a variety of means including a piston pump, a multiple action pump charging an accumulator via a check valve, a canister of compressed gas or a canister of propellant such as HFA. The reservoir 21 has a compressed gas outlet orifice 22 defined by a tube 23 terminating in a seat 24 through which gas may pass from the reservoir 20 via a servo chamber 25 and out of the assembly 20 through an exit orifice 26 to drug aerosolising means via a drug entrainment device (not shown). A valve member 27 is associated with the outlet orifice 21 to selectively permit or prevent the flow of compressed gas from the reservoir 21 into the servo chamber 25.

The valve member 27 comprises a flexible diaphragm 28 which extends across the end of the tube 22. A central region 29 of the diaphragm contacts the seat 24 to make a seal therewith when the valve is closed. It will be appreciated that only a relatively small central region 29 of the underside of the diaphragm 28 will be exposed to the effects of the pressure acting against it due to the source of compressed gas in the reservoir 20. The size of this region depends on the internal cross-sectional area of the tube 23.

The diaphragm 28 is located within and extends between the walls of a housing 30 to define a space or primary chamber 31 above the diaphragm 28, for reasons that will now be described.

It will be appreciated that when the reservoir 21 is pressurised to a pressure Pᵣₑₛ, a pressure force will be acting over the central region 29 of the diaphragm 28 which will tend to cause the diaphragm 28 to lift off the seat 24 and thus allow the gas to escape from the reservoir 21. To counteract this pressure force against the central region 29 of the diaphragm 28, the primary chamber 31 is also pressurised to a pressure Pₚ such that the force acting against the opposite side of the diaphragm 28 is sufficient to hold the central region 29 against the seat 24 and therefore keep the valve closed. The sealing force that must be generated by the pressure Pₚ in the primary chamber 31 which is sufficient to keep the valve closed is the sum of the seat force Fₛ of the diaphragm 28 against the seat 24 and the force Fₚ due to the pressure Pᵣₑₛ acting on the diaphragm 28 over the central region 29 of the diaphragm 28. Typically, the primary chamber 31 only needs to be pressurised to the same pressure as the reservoir 21, i.e. Pₚ=Pᵣₑₛ to keep the valve closed. This is because the pressure Pₚ acts over a much greater surface area of the diaphragm 28 than does the pressure Pᵣₑₛ.

The diameter of the tube 23 may be sufficiently large so as not to impede flow once the diaphragm 28 is open. The cross-sectional area of the tube 23 is limited only by needing to be smaller than the total cross sectional area of the diaphragm 28 so that the net force acting on the diaphragm is sufficient to ensure that its central region 29 seals against the valve seat 24, i.e. net force>seat force Fₛ.

To open the valve, it is necessary to lift the diaphragm 28 so that the seal is broken between the central region 29 of the diaphragm 26 and the seat 24. To do this, the diaphragm 28 can be lifted using a mechanical device (not shown). It will be appreciated that once the diaphragm 28 has been unseated, the pressure Pᵣₑₛ will now act over the whole of the underside of the diaphragm 28 rather than just the central region 29 thereof. As a result, the sealing force required to keep the valve closed and the force due to the pressure in the chamber 31 acting over the upper side of the diaphragm 28 will be equalised. As the net force now acting on the diaphragm 28 is zero, the valve opens rapidly.

To reset the valve by moving the diaphragm 28 back to its original closed position in which it locates against the seat 24, the primary chamber 31 is pressurised before the reservoir 20 so that the net force on the diaphragm 28 exceeds the required seat force between the central region 29 of the diaphragm 28 and the seat 24.

A first modified version of the assembly described with reference to FIG. 2 is shown in FIG. 3. In this arrangement, advanced pressurisation of the primary chamber 31 is rendered unnecessary as a biasing means, such as a spring 29, is disposed between the diaphragm 28 and the housing 30 and serves to bias the central region 29 of the diaphragm 28 against the seat 24 thereby making the valve self-resetting.

A second modified version of the assembly described with reference to FIG. 2 is shown in FIG. 4. In this arrangement, the diaphragm 26 is lifted from its seat 23 to open the valve by allowing pressure in the chamber 31 to decay to a point at which the force Fₚ due to the pressure acting on the diaphragm 28 is no longer sufficient to hold the central region 29 of the diaphragm 28 against the seat 24. Preferably, the pressure is allowed to decay by opening a port 32 in the housing 30 to communicate the chamber 31 to the atmosphere. This embodiment is particularly advantageous because the reservoir pressure Pᵣₑₛ acts to force the diaphragm 28 open therefore the discharge from the reservoir 21 is particularly rapid.

Although a mechanical device can be provided for opening and closing the port 32, the modified version of FIG. 2 can be adapted so that the port opens in response to the user's inhalation, as will now be described with reference to FIG. 5. For this purpose, the assembly is provided with a secondary valve member 33 which may be a breath actuated diaphragm 34, a vane or piston (not shown) mounted in a second housing 35 in a similar manner to the first diaphragm 28. The breath actuated diaphragm 34 has a central region 36 which seals against a seat 37 formed at the end of a tube 38 which extends from an aperture 40 that communicates the primary chamber 31 with the underside of the central region 36 of the breath actuated diaphragm 34 to block the flow of air from the primary chamber 31 to a primary chamber dump port 39 which is open to atmosphere. The upper surface of the secondary diaphragm 34 is in communication with the mouthpiece 5 via an opening 38.

When a user inhales through the mouthpiece 5, the central region 36 of the breath actuated diaphragm 34 is lifted from its seat 37 due to the lower pressure created in the mouthpiece 5 which is transmitted to the upper surface of the breath actuated diaphragm 34 via the opening 38. When the breath actuated diaphragm 34 is unseated, the primary chamber 31 is opened to the atmosphere via the aperture 40, the tube 38 and the primary chamber dump port 39. When this occurs, the pressure in the primary chamber 31 reaches a threshold at which the diaphragm 28 lifts rapidly releasing the charge of compressed gas from the reservoir 21 through the servo chamber 25 and the exit orifice 26 to deliver the dose of medicament via an airflow conduit 41 to a drug entrainment device and aerosolising means 43. It will be appreciated that when the breath actuated diaphragm 34 is lifted from its seat 37 when the user inhales, the pressure of the gas in the primary chamber will then act over the whole of the cross-sectional area of the underside of the breath actuated diaphragm rather than just over the central region 36. The pressure of the air in the primary chamber 31 therefore assists the breath actuated diaphragm 34 to open.

A biasing means such as a spring 44 acts against the breath actuated diaphragm 34 so that when the charge of gas in the primary chamber 31 has discharged, the breath actuation diaphragm 34 is automatically returned to the closed position by the spring 44. This arrangement allows the breath actuation diaphragm 34 to be self-resetting without the need for a separate resetting action by the user.

It will be appreciated that the valve uses a servo type action. When the diaphragm 28 is opened to a certain extent, high pressure air from the reservoir 21 floods the servo chamber 25 below the diaphragm 28 which then empties via the downstream drug entrainment and aerosolising means 43. If the flow resistance of the downstream entrainment device and aerosolising means 43 is much greater than that of the tube 22, the pressure in the servo chamber 25 will rapidly become almost equal to the reservoir pressure 21. This pressure acts on the underside of the diaphragm 28 and holds it open whilst the reservoir 21 is discharged.

It has been found by the inventors that the diameter of the chamber dump port 39 needs to be sufficiently large to facilitate rapid discharge of the primary chamber 31. If the primary chamber 31 is too small, the breath actuated diaphragm 34 can “bounce” or “flutter” causing the primary chamber 31 to discharge in stages compromising the efficiency of the inhaler. The cross-sectional area of the chamber dump port 39 should be greater than 0.15 mm² and should preferably be between 0.15 mm² and 0.75 mM². In a most preferable embodiment, the cross-sectional area of the chamber dump port 37 is 0.4 mm². If the dump port 39 has a cross-sectional area less than 0.15 mm², a delay is introduced between movement of the second diaphragm and the opening of the main valve diaphragm 26. Such a delay is undesirable, although if the dose is to be delivered later during an inhalation by the user, the dump port 39 could be designed so as to introduce a desired delay.

Although the chamber 31 can be provided with its own means to enable it to be pressurised, it is particularly desirable to use the means for charging the reservoir 21 to also charge the chamber 31. This can be achieved by, for example, incorporating a port (not shown) communicating the chamber 31 with the reservoir 21 which is closed prior to actuation of the valve.

The presence of a port between the reservoir 21 and the chamber 31 also prevents premature firing of the valve in the event of a leak from between the breath actuated diaphragm 34 and its seat 37 which can be caused due to, for example, imperfect sealing as a result of dirt ingress therebetween. As the diaphragm 28 is designed to open when the pressure difference between the primary chamber 31 and the reservoir 21 drops below a particular threshold, the possibility exists that a leak could cause the valve to open prematurely wasting the drug dose. However, it has been found that the diaphragm 28 will not servo open if the pressure is reduced sufficiently slowly and will instead open fractionally to allow gas to escape so that the reservoir pressure will drop in proportion to the slowly decreasing pressure in the chamber 31.

The assembly may be additionally provided with a control orifice (not shown) communicating the primary chamber 31 with the reservoir 21 so that any pressure drop in the chamber 31 due to a leak therein which is smaller than the control orifice constriction will be topped up from the reservoir 21.

Reference will now be made to the breath acutated valve module 50 forming part of an actual dry powder inhaler according to the invention which is illustrated in FIGS. 6 to 9. The breath actuated valve module 50 works as described with reference to FIGS. 2 to 5 and so like components will be referred to by the same reference numerals for ease of understanding.

A perspective view of the breath actuated valve module is shown in FIG. 6 and comprises an upper casing part 53 mounted on a lower casing part 54 using screws 55. The exit 26 through which the compressed air flows from the module to the aerosolising nozzle via the drug entrainment device can be seen, as can a connector 56 which connects the valve module 50 to the mouthpiece and through which the breath actuated diaphragm is controlled in response to inhalation by a user.

FIG. 7 illustrates a top plan view of the module 50 shown in FIG. 6 and FIGS. 8 and 9 illustrate two cross-sections taken along the lines A-A and B-B respectively. The cross-sectional illustrations show the outlet orifice 22 from the reservoir 21 and the tube 22 with the diaphragm 28 seated against the valve seat 28. The primary chamber 31 extends across the module and discharge of the compressed air from this chamber 31 through the chamber dump port 39 is selectively prevented by the breath actuated diaphragm 34 which is located against the valve seat 37 at the end of tube 38.

Many modifications and variations of the invention falling within the terms of the appended claims will be apparent to those skilled in the art and the foregoing description should be regarded as a description of the preferred embodiments only.

1. A dry powder inhaler for delivering a dose of medicament for inhalation by a user, including a drug entrainment device and a valve actuable by a user to cause pressurised gas to flow through a dose of medicament disposed in the drug entrainment device to entrain said dose in the gas, the valve comprising a valve member configured such that, in a first mode, pressurised gas biases the valve member into an open state to allow the flow of gas through the valve and, in a second mode, pressurised gas biases the valve member into a closed state to prevent the flow of gas through the valve.

2. An inhaler according to claim 1, wherein the valve is configured such that pressurised gas acts over both sides of the valve member when it is in the closed state.

3. An inhaler according to claim 2, wherein the valve is configured such that pressurised gas acts over a larger cross-sectional area of one side of the valve member than the pressurised gas acting over the other side of the valve member to keep the valve member in the closed state.

4. An inhaler according to claim 3, wherein the valve is configured such that the pressure acting over each side of the valve member is substantially the same when the valve member is in the closed state.

5. An inhaler according to claim 1, wherein the valve is configured such that the valve member moves from the closed state to the open state in response to a change in pressure of the gas acting on one side of the valve member relative to the pressure acting on the other side of the valve member.

6. An inhaler according to claim 5, comprising a reservoir for pressurised gas and a valve orifice for the passage of pressurised gas from the reservoir through the drug entrainment device, a first side of the valve member forming a seal with the valve orifice when in the closed state such that pressurised gas in said reservoir acts over only a portion of said first side of the valve member defined by the cross-sectional area of the valve orifice.

7. An inhaler according to claim 6, wherein the valve orifice is located at the mouth of a tube in communication with the reservoir, the tube including a valve seat at the end thereof for cooperation with said first side of the valve member to form a seal therewith when the valve member is in the closed state.

8. An inhaler according to claim 7, wherein the valve is configured such that when the seal between the first side of the valve member and the valve seat is broken, the pressure of the gas in the reservoir acts over substantially the entire surface of the first side of the valve member to bias the valve member into the open state.

9. An inhaler according to claim 6, comprising a mechanism which is arranged to bias the valve member into a closed state when the pressure of the gas in the reservoir has been discharged through the valve.

10. An inhaler according to claim 10, wherein said mechanism comprises a spring.

11. An inhaler according to claim 1 comprising a mechanism arranged to discharge the pressure that biases the valve member into the closed state to cause the valve member to move from the closed to the open state.

12. An inhaler according to claim 11, wherein the valve includes a primary chamber in which pressure to bias the valve member into the closed state is generated and said mechanism comprises a discharge port in the primary chamber.

13. An inhaler according to claim 12, wherein the discharge port selectively opens to atmosphere.

14. An inhaler according to claim 13, wherein the discharge port opens to atmosphere via breath actuation.

15. An inhaler according to claim 14, wherein the valve includes a secondary valve member which is movable, in response to inhalation by a user, from a first closed position in which the discharge port is not in communication with the primary chamber to prevent discharge of the primary chamber to the atmosphere, into a second open position in which the discharge port is in communication with the primary chamber to discharge the primary chamber to the atmosphere.

16. An inhaler according to claim 15, wherein the secondary valve member is configured such that the pressure in the primary chamber acts over a smaller cross-sectional area of a first side of the secondary valve member than the cross-sectional area of the other side of the valve member over which atmospheric pressure acts, when the secondary valve member is in the closed position.

17. An inhaler according to claim 15, comprising a biasing mechanism, the biasing mechanism biasing the secondary valve member into a closed state when the pressure of the gas in the primary chamber has discharged through the discharge port.

18. An inhaler according to claim 17, wherein the biasing mechanism comprises a spring.

19. An inhaler according to claim 15, wherein the valve is configured such that the secondary valve member is in the closed position, to prevent the discharge of pressure from the primary chamber to the atmosphere, when the pressure acting over each side of the secondary valve member is substantially the same.

20. An inhaler according to claim 15, wherein the secondary valve member is a flexible diaphragm.

21. An inhaler according to claim 12, comprising a source of pressurised gas or air, said source charging the reservoir.

22. An inhaler according to claim 21, wherein the source of pressurised gas or air also charges the primary chamber.

23. An inhaler according to claim 22, wherein a conduit communicates the reservoir with the primary chamber.

24. An inhaler according to claim 21, wherein the source of pressurised gas or air comprises a device selected from the group consisting of: a piston pump, a multiple action pump charging an accumulator via a check valve, a canister of compressed gas and a canister of propellant.

25. An inhaler according to claim 1, wherein the valve member is a flexible diaphragm.

26. (canceled)

27. An inhaler according to claim 24, wherein said propellant is HFA.
